(19) United States
(12) Patent Application Publication
(10) Pub. No.: US 2019/0275002 A1
(43) Pub. Date: Sep. 12, 2019

(54) METHOD AGAINST INFLUENZA A VIRAL INFECTION WITH TRYPTOPHAN AND ARGININE

(71) Applicants: Chang Gung Memorial Hospital, Linkou, Taoyuan City (TW); Chang Gung University, Taoyuan City (TW); National Taiwan University, Taipei City (TW)

(72) Inventors: Chia-Ni LIN, Taipei City (TW); Ching-Hwa TSAI, New Taipei City (TW); Sue-Jane LIN, Taichung City (TW)

(21) Appl. No.: 16/054,542

(22) Filed: Aug. 3, 2018

(30) Foreign Application Priority Data
Mar. 12, 2018 (TW) 107108208

(51) Int. Cl. A61K 31/405; A61K 9/00; A61P 31/16

(52) U.S. Cl. CPC A61K 31/405; A61K 9/0053; A61K 31/198; A61P 31/16; A61K 2300/00; A61K 9/0019

(57) ABSTRACT

Disclosed herein is a method against influenza A viral infection, including administering to a subject in need thereof tryptophan and arginine.

### CROSS-REFERENCE TO RELATED APPLICATION

This application claims priority of Taiwanese Patent Application No. 107108208, filed on Mar. 12, 2018.

### FIELD

The disclosure relates to a method against influenza A viral infection with tryptophan and arginine.

### BACKGROUND

Influenza, commonly referred to as flu, is a contagious acute respiratory disease caused by infection with an influenza virus. Seasonal influenza epidemics occur every year, mainly during winter in both Northern and Southern Hemispheres. The influenza viruses that affect humans can be divided, depending on the differences of nucleoproteins and matrix proteins of the viruses, into three types: A, B and C. Among these influenza viruses, influenza A virus causes serious consequences on human health.

Influenza A virus is an RNA virus belonging to the genus Influenza virus A of the family Orthomyxoviridae. Such virus can be further classified into different subtypes, e.g., H1, H2, H3, H5, H7 and H9, based on the distinction of two large glycoproteins located on the surface of the virus, hemagglutinin (HA) and neuraminidase (NA).

Influenza A virus spreads mainly through droplets in the airborne route (i.e., a person inhales aerosols produced by coughing, sneezing or spitting of an infected person). A subject, when infected with influenza A virus, normally has symptoms including fever, chills, sore throat, muscle pain, headache and fatigue. Without proper treatment given, serious health complications (such as pneumonia, otitis media, encephalitis and pericarditis) might occur, especially in high-risk groups such as young children, the elderly, health care workers, and people with chronic illnesses (e.g. diabetes, asthma, heart diseases, etc.) or those who are immuno-compromised among others.

Currently, clinical methods for treating influenza A viral infection include symptomatic treatment, supportive therapy and antiviral therapy. Among these methods, antiviral therapy is carried out by administering antiviral agents effective in suppressing specific functions of viral proteins that are essential for virus replication and infection. In general, the antiviral agents used against influenza A viral infection are classified into two major types: (1) neuraminidase inhibitors, including oseltamivir (trade name: Tamifiu®), zanamivir (trade name: Relenza®) and peramivir (trade name: Rapiacta®); and (2) M2 ion channel inhibitors, including amantadine and rimantadine. However, therapeutic effect of these antiviral agents on the treatment of influenza A viral infection is far from ideal and might even cause serious side effects and drug resistance inpatients. Therefore, a committed goal of this field is to develop antiviral agents or drugs that can effectively treat influenza A viral infection without causing undesirable side effects.

It has been reported that, amino acids and their analogues may be effective in treating and/or against influenza A viral infection. As described in Ikeda K. et al. (2010), Exp. Ther. Med., 1:251-256, a strain of influenza A virus (strain A/Aichi/2/1968 H3N2) was incubated with a 0.7 M arginine-containing solution at a respective one of different pH values (i.e., pH 4.5, 5.0 and 5.5) on ice for 30 to 60 minutes. The resultant culture medium was collected and inoculated into a Madin-Darby canine kidney (MDCK) cell line. Then, the number of infectious viruses was measured by plaque assay. The experimental results showed that the arginine-containing solution at pH 4.5 exhibited the most potent virus inactivation effect, and that this effect decreased rapidly with increase in the pH value of the solution. Specifically, no virus inactivation effect was detected when influenza A virus was cultured at pH 5.5. Based on this finding, Ikeda K. et al. deduced that an acidic arginine solution can be used in the treatment of influenza A viral infection.

As reported in Akaike T. et al. (1996), Proc. Natl. Acad. Sci. USA, 93:2448-2453, mice were infected with a strain of influenza A virus (strain A/Kumamoto/Y5/67 H2N2). Then, the infected mice were administered with L-N^{G}-monomethyl arginine citrate (L-NMMA), a type of nitric oxide synthase (NOS) inhibitor, and their survival rate was observed for 16 days. In addition, on Day 7 after the viral infection, some of the mice were sacrificed, and the lungs thereof were subjected to an electron spin resonance measurement, so as to detect the NO-hemoglobin signal. The experimental results showed that, administration of L-NMMA can inhibit the production of NO-hemoglobin in the lungs of the infected mice, thus effectively increasing the survival rate of these mice. Akaike T. et al. thus inferred that suppression of NO and/or O₂⁻, which may be the most important pathogenic factors for the influenza virus-induced pneumonia, may be beneficial to the viral-infected mice.

As mentioned in Fox J. M. et al. (2013), J. Gen. Virol., 94:1451-1461, C57BL/6 mice were fed with drinking water containing a indoleamine-2,3-dioxygenase (IDO) inhibitor, i.e., 1-methyl-tryptophan (1-MT), for 3 days. The mice were then infected intranasally with a strain of influenza A virus (strain A/Hong Kong/X31/68 H3N2), and continued to be fed with the drinking water after virus infection. Afterwards, bronchoalveolar lavage (BAL) fluid was collected from the lungs of the infected mice, and a single-cell suspension was isolated therefrom. The thus obtained single-cell suspension was incubated with antibodies capable of detecting virus-specific T-cells, followed by subjecting the suspension to intracellular cytokine staining (ICS) combined with flow cytometry for phenotyping and quantification of lymphocyte population. The experimental results showed that, in the lungs of the influenza A virus-infected mice, the number of activated and functional CD4⁺ T-cells, influenza-specific CD8⁺ T-cells and effector memory cells increased significantly. It was inferred by Fox J. M. et al. that, the inhibition of IDO activity by 1-MT can increase T-cell responses and thus enhances aspects of the adaptive immune response to influenza virus infection.

### SUMMARY

Therefore, an object of the present disclosure is to provide a method against influenza A viral infection that can alleviate at least one of the drawbacks associated with the prior art.

According to the present disclosure, a method against influenza A viral infection includes administering to a subject in need thereof tryptophan and arginine.

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it should be clearly understood that the word “comprising” means “including but not limited to”, and that the word “comprise” has a corresponding meaning.

Unless otherwise defined, all technical and scientific terms used herein have the meaning as commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

In the development of drugs that can be used to treat influenza A viral infection, the applicants unexpectedly found that use of tryptophan and arginine can significantly inhibit the replication of influenza A virus, and hence can reduce the viral load of influenza A virus in mice lungs. The combination of tryptophan and arginine is thus expected to be effective against influenza A viral infection.

Therefore, the present disclosure provides a method against influenza A virus infection, which includes administering to a subject in need thereof tryptophan and arginine.

As used herein, the term “against influenza A viral infection” or “anti-influenza A viral infection” means prevention of infection by influenza A virus, suppression of influenza A virus replication, and/or treatment and/or prevention of infectious diseases caused by influenza A virus.

As used herein, the term “administration” or “administering” means introducing, providing or delivering a pre-determined active ingredient to a subject by any suitable routes to perform its intended function.

As used herein, the term “administered simultaneously” or “simultaneous administration” means that a first active ingredient and a second active ingredient are administered concurrently.

As used herein, the term “administered sequentially” or “sequential administration” means that there is a predetermined time interval between the administration of a first active ingredient and the administration of a second active ingredient, such that the pharmacological effects of the administered active ingredients overlap in time.

According to this disclosure, the ratio of tryptophan and arginine may be adjustable with actual application, routes of administration, etc., in order to achieve the best effect against influenza A viral infection. In an exemplary embodiment, tryptophan and arginine may be administered in a molar ratio of 3:5.

According to this disclosure, influenza A virus may be of at least one selected from H1, H2, H3, H5, H7 and H9 subtypes.

Examples of the H1 subtype of influenza A virus may include, but are not limited to, H1N1, H1N2, H1N3, H1N4, H1N5, H1N6, H1N7, H1N8, H1N9 and H1N10.

Examples of the H2 subtype of influenza A virus may include, but are not limited to, H2N1, H2N2, H2N3, H2N4, H2N5, H2N6, H2N7, H2N8, H2N9 and H2N10.

Examples of the H3 subtype of influenza A virus may include, but are not limited to, H3N1, H3N2, H3N3, H3N4, H3N5, H3N6, H3N7, H3N8, H3N9 and H3N10.

Examples of the H5 subtype of influenza A virus may include, but are not limited to, H5N1, H5N2, H5N3, H5N4, H5N5, H5N6, H5N7, H5N8, H5N9 and H5N10.

Examples of the H7 subtype of influenza A virus may include, but are not limited to, H7N1, H7N2, H7N3, H7N4, H7N5, H7N6, H7N7, H7N8, H7N9 and H7N10.

Examples of the H9 subtype of influenza A virus may include, but are not limited to, H9N1, H9N2, H9N3, H9N4, H9N5, H9N6, H9N7, H9N8, H9N9 and H9N10.

According to this disclosure, tryptophan and arginine may be, separately or together, prepared into a pharmaceutical composition.

According to this disclosure, the pharmaceutical composition may be formulated into a dosage form suitable for parenteral or oral administration using technology well-known to those skilled in the art. Examples of the dosage form include, but are not limited to, injections (e.g., sterile aqueous solutions or dispersions), sterile powder, tablets, troches, lozenges, capsules, dispersible powder, granule, solutions, suspensions, emulsions, syrup, elixirs, slurry and the like.

In certain embodiments, the pharmaceutical composition may be administered by parenteral routes selected from the group consisting of intraperitoneal injection, intrapleural injection, intramuscular injection, intravenous injection, intraarterial injection, intraarticular injection, intrasynovial injection, intrathecal injection, intracranial injection and sublingual administration. In an exemplary embodiment, the pharmaceutical composition may be made into a dosage form suitable for intravenous injection.

In certain embodiments, the pharmaceutical composition may be made into a dosage form suitable for oral administration.

According to this disclosure, the pharmaceutical composition may further include a pharmaceutically acceptable carrier that is widely employed in the art of drug-manufacturing. Examples of the pharmaceutically acceptable carrier may include, but are not limited to, solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, and the like. The choice and amount of the pharmaceutically acceptable carrier are within the expertise of those skilled in the art.

In certain embodiments, the pharmaceutically acceptable carrier may include a solvent selected from the group consisting of normal saline, phosphate buffered saline (PBS), a sugary solution, an aqueous solution containing alcohol, and combinations thereof.

In an exemplary embodiment, the two active ingredients of this disclosure (i.e., tryptophan and arginine) may be combined and administered in a single dosage form (i.e., fixed-dose combination).

In certain embodiments, the two active ingredients of this disclosure may be administered as two separate dosage forms, each containing one of the active ingredients. The two separate dosage forms may be administered substantially concurrently, or may be administered alternately or sequentially on the same or separate days. That is, tryptophan and arginine of this disclosure may be administered simultaneously, sequentially or separately.

As used herein, the term “administered separately” or “separate administration” means that there is a predetermined time interval between the administration of a first active ingredient and the administration of a second active ingredient, such that when the second active ingredient is administered to a subject thereof, the administered first active ingredient is no longer present in a therapeutically effective amount in the blood of the subject.

According to this disclosure, the dose and frequency of administration of tryptophan and arginine may vary depending on the following factors: the severity of the illness to be treated, routes of administration, and age, physical condition and response of the subject to be treated. In general, tryptophan and arginine may be administered in a single dose or in several doses, and may be orally or parenterally administered.

This disclosure will be further described by way of the following examples. However, it should be understood that the following examples are solely intended for the purpose of illustration and should not be construed as limiting the disclosure in practice.

### EXAMPLES

### Example 1. Evaluation for the Effect of the Combination of Tryptophan and Arginine Against Influenza A Virus

### Experimental Materials:

### 1. Source and Cultivation of Cell Lines:

Human embryonic kidney 293T (293T) cells and Madin-Darby canine kidney (MDCK) cells used in the following experiments were obtained from Chang Gung Memorial Hospital, Linkou, Taiwan. Each of the cell lines was incubated in a 10 cm Petri dish containing Dulbecco's modified Eagle medium (DMEM; HyClone) supplemented with 10% fetal bovine serum (FBS; HyClone), 100 units/mL penicillin, 100 μg/mL streptomycin, 0.1 mM non-essential amino acids and 2 mM L-glutamine, followed by cultivation in an incubator with culture conditions set at 37° C. and 5% CO₂. When the cultured cells reached about 100% confluency, the culture medium was removed and the cells were washed once with phosphate buffered saline (PBS). Then, trypsin-EDTA was added to detach the cells from the bottom surface of the Petri dish. Afterwards, a fresh culture medium was added to neutralize trypsin activity and the culture medium was repeatedly pipetted to completely disperse the cells. The resulting cell suspension was then dispensed into a new Petri dish, and then was subcultured in the incubator with the abovementioned culture conditions.

### 2. Experimental Mice:

C57BL/6J mice (8 weeks old, with a body weight of about 22 to 25 g) used in the following experiments were purchased from Jackson Laboratory (Bar Harbor, Me., USA). All the experimental mice were housed in an animal room with an independent air conditioning system under the following laboratory conditions: specific pathogen-free standard, an alternating 12-hour light and 12-hour dark cycle, a temperature maintained at 22±2° C., and a relative humidity maintained at 65±5%. The mice were provided with water and feed ad libitum. All the experimental procedures for the experimental mice were carried out according to the guidelines of the Institutional Animal Care and User Committee of Chang Gung University, Linkou, Taiwan.

### 3. Preparation of Influenza a Virus:

A strain of influenza A virus (strain A/Puerto Rico/8/1934/H1N1, hereinafter referred to as PR8 virus strain) used in the following experiments was prepared using a reverse genetics system according to a method described in Lin S. J. et al. (2014), J. Biomed. Sci., 21:99, doi: 10.1186/s12929-014-0099-6.

Briefly, 8 plasmids (namely pPolI-PR8-PB2, pPolI-PR8-PB1, pPolI-PR8-PA, pPolI-PR8-HA, pPolI-PR8-NP, pPolI-PR8-NA, pPolI-PR8-M and pPol-PR8-NS, which were provided by Prof. Shin-Ru Shih from the Research Center for Emerging Viral Infections at Chang Gung University, Linkou, Taiwan) were mixed thoroughly with a TranslT-LT1 transfection reagent (Mirus Bio LLC) to obtain a transfection mixture. The 293T cells obtained above were added with the transfection mixture and then incubated for 48 hours. Afterwards, the culture medium was harvested and inoculated into an allantoic cavity of a 10-day-old embryonated chicken egg, followed by incubation for 2 hours. The inoculated egg was placed at 4° C. overnight. The fluid in the allantoic cavity of the egg was collected and subjected to centrifugation at 200×g for 10 minutes at 4° C. The thus obtained supernatant was collected for calculating viral plaques therein using a plaque assay. Based on the plaque assay, it was deduced that the obtained supernatant had a viral titer of 10⁸-10¹⁰ plaque-forming units (pfu)/mL. Afterwards, an appropriate amount of the supernatant was mixed with 30 μL of PBS to obtain a PR8 virus solution (with a virus amount of 200 pfu). The PR8 virus solution was stored in a freezer at −80° C. for further experiment.

4. Types and sources of conventional NOS, IDO and arginase-1 inhibitors used in the following experiment are shown in Table 1.

TABLE 1

| Inhibitors | Sources |
|---|---|
| L-NG-monomethyl arginine citrate | Sigma-Aldrich |
| (L-NMMA), serving as a nitric oxide | |
| synthase (NOS) inhibitor | |
| 1-methyl-tryptophan (1-MT), serving | Enzo Life |
| as an indoleamine-2,3-dioxygenase | Sciences |
| (IDO) inhibitor | |
| N^{ω}-hydroxy-nor-L-arginine acetate | |
| (nor-NOHA), serving as an arginase-1 | |
| inhibitor | |

### 5. Preparation of Amino Acid Solution:

An appropriate amount of tryptophan powder and arginine powder (both purchased from Sigma-Aldrich) were mixed together in 50 mL of PBS to obtain an amino acid solution containing 1.5 mM tryptophan and 2.5 mM arginine.

### 6. Preparation of 0.4% Agarose Medium:

0.8% agarose (in PBS) was mixed with serum-free 2-fold concentrated DMEM in a ratio of 1:1 (v/v) to obtain a 0.4% agarose medium.

### Experimental Procedures:

### A. Inoculation of PR8 Virus Strain

The C57BL/6J mice were randomly divided into a control group (n=17), four inhibitor groups [i.e., inhibitor group 1 (n=12), inhibitor group 2 (n=11), inhibitor group (n=14) and inhibitor group 4 (n=14)], and an experimental group (n=15). The drinking water for the mice of each inhibitor group was added with the respective conventional inhibitor(s) as shown in Table 2. The drinking water for the mice of the control group and the experimental group was not added with any inhibitor.

TABLE 2

| | Inhibitors | | |
|---|---|---|---|
| | L-NMMA | nor-NOHA | 1-MT |
| Groups | (5 μM) | (5 μM) | (5 μM) |
| Inhibitor group 1 | + | − | − |
| Inhibitor group 2 | − | + | − |
| Inhibitor group 3 | − | − | + |
| Inhibitor group 4 | + | + | + |

The mice of each group were fed for 3 days with the respective drinking water mentioned above. Afterwards, the mice of the four inhibitor groups and the experimental group were infected with the PR8 virus solution via intranasal instillation at a dosage of 200 pfu per mouse. The mice of the control group received no infection.

### B. Administration of Amino Acid Solution Containing Tryptophan and Arginine

After the virus inoculation described in the above section A, the mice in each group were fed for 7 days. Specifically, the drinking water for each of the inhibitor groups and the control group was prepared according to the procedures described in the above section A, while the drinking water for the experimental group was added with the amino acid solution (containing 1.5 mM tryptophan and 2.5 mM arginine) in a ratio of 100:1 (v/v). In addition, on the second, fourth and sixth days after virus inoculation, the mice of the experimental group were also subjected to intraperitoneal injection with 1 mL of the amino acid solution.

On the seventh day after virus inoculation, the mice in each group were sacrificed by servical dislocation. The lung tissues of each group of mice were harvested, and 2 mL of DMEM was added to grind the lung tissues for homogenization. Afterwards, the resultant lung homogenized fluid was subjected to a ten-fold serial dilution using PBS, so as to obtain three diluted solutions each having a respective dilution factor (i.e., 10¹, 10² and 10³ times). Then, each of the diluted solutions was treated with an appropriate amount of trypsin to obtain a final trypsin concentration of 0.0005%, followed by subjecting the solutions to the following plaque assay.

### C. Plaque Assay

The MDCK cells were seeded at 1×10⁶ cells per well into 6-well plates containing DMEM, and were cultured in an incubator (37° C. and 5% CO₂) for 24 hours. Then, the cultured MDCK cells were added with 500 μL of a respective one of the three diluted solutions of each group, followed by conducting incubation at 37° C. for 1 hour in order for the virus to be adsorbed into the cells. Afterwards, the culture medium in each well was removed, and a preheated 0.4% agarose medium was added to each cell and allowed to overlay the cultured MDCK cells therein. After the agarose medium had solidified, the plates were placed in an incubator (37° C. and 5% CO₂) for 2 days. Then, 3 mL of 10% formalin was added to each well to fix the cells for 1 hour. After removal of the formalin and the agarose medium, the fixed cells were dyed with 1% (w/v) crystal violet (Sigma-Aldrich) for 2 minutes. Afterwards, distribution of the viral plaques in the cultured MDCK cells with respect to each group was analyzed by visual observation, and the dilution factor of the diluted solution that would form approximately 15 to 20 single virus plaques was selected. Viral load in the mice lungs of each group was calculated by substituting the selected dilution factor and the number of viral plaques formed thereby into the following formula (1):

A=(B/0.5)×C×2 (1)

wherein: A=viral load (pfu)

B=number of viral plaques counted

C=dilution factor

### Results:

The average viral load of each group is shown in Table 3.

TABLE 3

| Group | Average viral load (pfu) |
|---|---|
| Control group | 8.05 × 10³ |
| Inhibitor group 1 | 4.30 × 10³ |
| Inhibitor group 2 | 2.47 × 10³ |
| Inhibitor group 3 | 4.37 × 10³ |
| Inhibitor group 4 | 8.94 × 10³ |
| Experimental group | 0.73 × 10³ |

As shown in Table 3, the average viral load of the experimental group, as well as those of the inhibitor groups 1, 2 and 3, was significantly lower than that of the control group, while the average viral load of inhibitor group 4 was similar to that of the control group. In addition, the average viral load in the experimental group showed a significant decrease of virus compared to those in the inhibitor groups 1, 2, 3 and 4. These experimental results indicated that use of tryptophan and arginine can effectively inhibit the replication of influenza A virus, which in turn reduces the viral load of influenza A virus in the mice lungs. Therefore, the combination of tryptophan and arginine is expected to be effective against influenza A viral infection.

All patents and literature references cited in the present specification as well as the references described therein, are hereby incorporated by reference in their entirety. In case of conflict, the present description, including definitions, will prevail.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method against influenza A viral infection, comprising administering to a subject in need thereof tryptophan and arginine.

2. The method of claim 1, wherein the tryptophan and arginine are administered simultaneously.

3. The method of claim 1, wherein the tryptophan and arginine are administered sequentially.

4. The method of claim 1, wherein the tryptophan and arginine are administered separately.

5. The method of claim 1, wherein the influenza A virus is at least one selected from the group consisting of H1, H2, H3, H5, H7 and H9 subtypes.

6. The method of claim 1, wherein the tryptophan and arginine are administered in a molar ratio of 3:5.

7. The method of claim 1, wherein the tryptophan and arginine are administered in a single dosage form.

8. The method of claim 1, wherein the tryptophan and arginine are administered in separate dosage forms.

9. The method of claim 1, wherein the tryptophan and arginine are parenterally administered.

10. The method of claim 1, wherein the tryptophan and arginine are orally administered.
